(19) United States
(12) Patent Application Publication
(10) Pub. No.: US 2024/0245537 A1
(43) Pub. Date: Jul. 25, 2024

(54) BIODEGRADABLE DRUG ELUTING STENT

(71) Applicant: Suzhou RongChuangJiaHe Medical Technology Co., Ltd., Suzhou (CN)

(72) Inventor: Shao-Liang Chen, Nanjing (CN)

(21) Appl. No.: 18/424,599

(22) Filed: Jan. 26, 2024

Related U.S. Application Data
(63) Continuation of application No. PCT/CN2022/103971, filed on Jul. 5, 2022.

(30) Foreign Application Priority Data
Jul. 28, 2021 (WO) PCT/CN2021/108897

(51) Int. Cl. A61F 2/86

(52) U.S. Cl. CPC A61F 2/86; A61F 2250/0067

(57) ABSTRACT

A biodegradable drug-eluting stent comprises a first non-biodegradable end segment, a biodegradable intermediate segment coupled to the first non-biodegradable end segment, and a second non-biodegradable end segment coupled to the biodegradable intermediate segment. The stent also comprises a plurality of connectors connecting the first and second end segments to the intermediate segment, each connector comprising a biodegradable connector arm coupled to and extending away from an end of the intermediate segment and a non-biodegradable arm receiver coupled to an end of the first or second end segment. The connector arm has an end tab and the arm receiver is configured to couple with the end tab. The end segments are drug-eluting and are configured to expand at a faster rate than that of the intermediate segment in response to an expansion force from an expandable balloon the stent is crimped over.

### CROSS-REFERENCE

This application is a continuation of International Application No. PCT/CN2022/103971 filed on Jul. 5, 2022, which claims priority to PCT Application No. PCT/CN2021/108897 filed on Jul. 28, 2021; each of which is incorporated herein by reference.

### BACKGROUND

The present disclosure relates to medical devices, systems, and methods, particularly stents and vascular scaffolds.

In medicine, a stent is a metal or plastic tube inserted into the lumen of an anatomic vessel or duct to keep the passageway open, and stenting is the placement of a stent. There is a wide variety of stents used for different purposes, from expandable coronary, vascular, and biliary stents, to simple plastic stents used to allow the flow of urine between kidney and bladder.

The most commonly used stents are coronary and vascular stents. Coronary stents are placed during a coronary angioplasty. The most common use for coronary stents is in the coronary arteries, into which a bare-metal stent, a drug-eluting stent, a bioabsorbable stent, a dual-therapy stent (combination of both drug and bioengineered stent), or occasionally a covered stent is inserted. Vascular stents are a common treatment for advanced peripheral and cerebrovascular disease. Common sites treated with vascular stents include the carotid, iliac, and femoral arteries.

Drug-eluting stents and bioresorbable stents have in recent times seen greater use and technical advancement. A drug-eluting stent (DES) is a peripheral or coronary stent (i.e., a scaffold) placed into narrowed, diseased peripheral or coronary arteries that slowly releases a drug to block cell proliferation. The release of the drug prevents fibrosis that, together with clots (i.e., thrombi), could otherwise block the stented artery, a process called restenosis. The stent is usually placed within the peripheral or coronary artery by an interventional cardiologist or interventional radiologist during an angioplasty procedure. A bioresorbable stent, also often called a bioresorbable scaffold, a biodegradable stent, or a naturally-dissolving stent, serves the same purpose as a stent, but is manufactured from a material that may dissolve or be absorbed in the body.

While drug-eluting stents have many advantages and, in some cases, have been proven to be superior to bare-metal stents, having lower rates of major adverse cardiac events, they are not free of all drawbacks. In at least some cases, the metal is permanently left in the blood vessel after drug release can cause thrombosis and, in at least some cases, necessitate a long period of antiplatelet therapy which increases the risk of bleeding for the patient. In at least some cases, if the patient in the future needs a surgical bypass surgery at the stented site, the stent can prevent suturing of any bridging blood vessel. Further, in at least some cases, heavy metal residues can be found in the controlled release membrane, which may be harmful to the patient.

Bioresorbable stents or vascular scaffolds can have the advantage over drug-eluting stents and bare-metal stents in that they may be degraded over time, for example, typically three years, which permits future bypass surgery and reduces the risk of thrombosis and heavy metal residues. Bioresorbable stents, however, may be disadvantaged in other areas. In at least some cases, in the process of biodegradation, the ends of the stent may be prone to collapse, leading to one or more ends of the stent being suspended in the vessel lumen and therefore increasing risk of thrombus formation. In at least some cases, bioresorbable stents are made of biodegradable materials that are too thick to allow such stents to be implanted in an overlapped or nested manner which may be needed for diffuse lesions or long lesions. Further, in at least some cases, bioresorbable stents are not applicable for bifurcation lesions where two stents are required, because such stents cannot be overlapped or nested with each other due to the relatively greater wall thickness of such stents.

For at least these reasons, improvements to stents, particularly drug-eluting stents and bioresorbable stents, are desired.

The following reference may be relevant: US20170181872A1, US20070288084A1, U.S. Ser. No. 10/932,928B2, U.S. Pat. No. 9,907,644B2, U.S. Pat. No. 9,326,870B2, U.S. Pat. No. 8,814,927B2, U.S. Pat. No. 8,603,154B2, U.S. Pat. No. 7,789,906B2, and WO2019138416A1.

### SUMMARY

The present disclosure provides biodegradable drug eluting stents which address at least some of the drawbacks of drug-eluting stents and bioresorbable stents described above. An exemplary stent may comprise a first non-biodegradable end segment, a biodegradable intermediate segment having a first end coupled to an end of the first non-biodegradable end segment, and a second non-biodegradable end segment having an end coupled to a second end of the biodegradable intermediate segment. The end segments may be drug-eluting and non-biodegradable. By providing non-biodegradable end segments, a plurality of such stents may be overlapped or nested, making such stents applicable for bifurcation lesions. The intermediate segment of the stent may degrade over time, leaving the non-biodegradable end segments in place. Should restenosis occur, such as where the biodegraded intermediate segment had been, a shorter stent may be implanted in its place.

The exemplary stent may also comprise a plurality of connectors connecting the first and second end segments to the intermediate segment. Each connector may comprise a biodegradable connector arm coupled to and extending away from the first or second end of the biodegradable intermediate segment and a non-biodegradable arm receiver coupled to the end of the first or second end segment. The biodegradable connector arm may have an end tab. The arm receiver may be configured to couple with this end tab. The connectors may provide mortise and tenon joints between the non-biodegradable end segments and the biodegradable intermediate segment, which may prevent the lateral ends of the biodegradable end segment from collapsing as its material degrades.

The first and second end segments may be configured to expand at a first rate in response to an expansion force. The intermediate segment may be configured to expand at a second rate in response to the expansion force. The first rate may be faster than the second rate. The first end segment may comprise at least one ring of struts arranged in a first non-biodegradable strut pattern. The second end segment may comprise at least one ring of struts arranged in a second non-biodegradable strut pattern. The intermediate segment may comprise at least one ring of struts arranged in a biodegradable strut pattern. The biodegradable strut pattern of the intermediate segment may be denser than the first and second non-biodegradable strut patterns of the first and second non-biodegradable end segments, which can thereby cause the differential expansion response of the end and intermediate segments. During expansion of the stent, typically by expansion of the balloon or other expandable member (such as a malecot) the stent is crimped over, the non-biodegradable end segments will typically expand first, which can facilitate a stabilization of the biodegradable intermediate segment. Such stabilization can prevent any displacement of the biodegradable intermediate segment and the connector parts coupled thereto, as well as prevent any damage to the mortise and tenon structure of the connectors.

Further, the non-biodegradable end segments are typically made of a metal or other material highly visible to fluoroscopy or other medical imaging. These segments can therefore facilitate the accurate delivery and positioning of the stent.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows a side view of an exemplary stent, according to embodiments herein.

FIG. 2 shows a side view of another exemplary stent, according to embodiments herein.

FIG. 3A shows a magnified side view of an illustration of a connector for end and intermediate segments of the stents described herein, according to embodiments.

FIG. 3B shows a section view of an embodiment of the connector of FIG. 3A.

FIG. 3C shows a section view of an embodiment of the connector of FIG. 3A.

FIG. 3D shows a magnified side view of a connector of the stent of FIG. 1.

FIG. 3E shows a magnified side view of a connector of the stent of FIG. 2.

FIG. 4 shows illustrations of stent segment patterns for the stents described herein, according to embodiments.

FIGS. 5A-5F show the expansion of an exemplary stent according to embodiments herein.

FIGS. 6A, 6B, 6C, and 6D show optical coherence tomography (OCT) images of blood vessels with a bare-metal stent (FIG. 6A), a drug-eluting stent (FIG. 6B), and a hybrid stent according to embodiments herein (FIGS. 6C-6D), respectively, immediately after deployment and at a one month follow-up (FIGS. 6A-6C) and a three month follow-up (FIG. 6D).

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain inventive embodiments herein contemplate numerical ranges. When ranges are present, the ranges include the range endpoints. Additionally, every sub range and value within the range is present as if explicitly written out. The term “about” or “approximately” may mean within an acceptable error range for the particular value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 10% of a given value. Where particular values are described in the application and claims, unless otherwise stated the term “about” meaning within an acceptable error range for the particular value may be assumed.

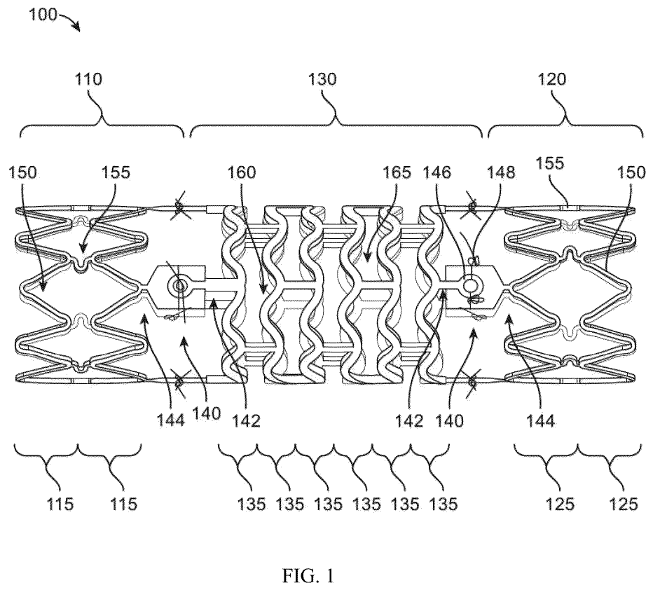

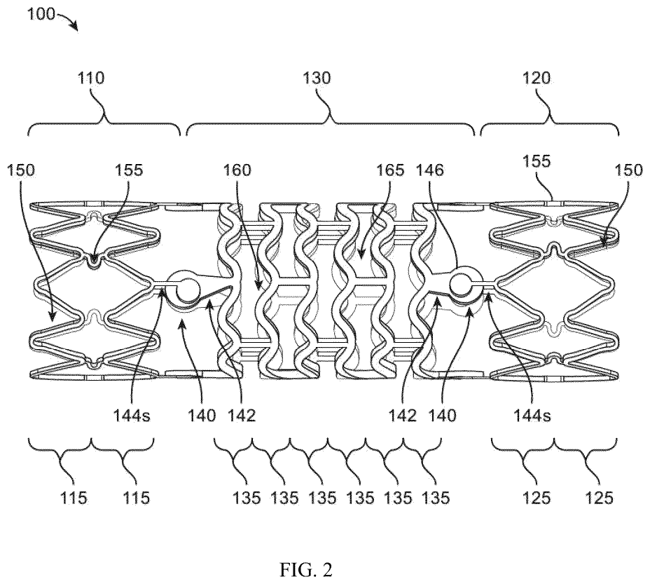

As shown in at least FIG. 1 and FIG. 2, an exemplary stent 100 according to embodiments herein may comprise a first non-biodegradable end segment 110, a biodegradable intermediate segment 130 having a first end coupled to an end of the first non-biodegradable end segment 110 and a second end, and a second non-biodegradable end segment 120 having an end coupled to the second end of the biodegradable intermediate segment 130.

The first end segment 110 and the second end segment 120 may be configured to expand at a first rate in response to an expansion force, for example, from a balloon 500 or other expandable member (such as a malecot) the stent 100 is crimped over. The intermediate segment 130 may be configured to expand at a second rate in response to the same expansion force, and the first rate may be faster than the second rate. During expansion of the stent, the non-biodegradable end segments 110, 120 will typically expand first, which can facilitate a stabilization of the biodegradable intermediate segment 130. Such stabilization can prevent any displacement of the biodegradable intermediate segment 130 and the connectors 140, as well as prevent any damage to the connectors 140.

The first end segment 110 comprises at least one ring 115 of struts arranged in a first non-biodegradable strut pattern. The second end segment 120 may comprise at least one ring 125 of struts arranged in a second non-biodegradable strut pattern. The intermediate segment 130 may comprises at least one ring of struts 135 arranged in a biodegradable strut pattern. The biodegradable strut pattern of the intermediate segment may be denser than the first and second non-biodegradable strut patterns of the first and second non-biodegradable end segments. This increased density may contribute at least in part to the increased stiffness of the intermediate segment 130 and its slower expansion in response to the same expansion forces and the first end segment 110 and the second end segment 140.

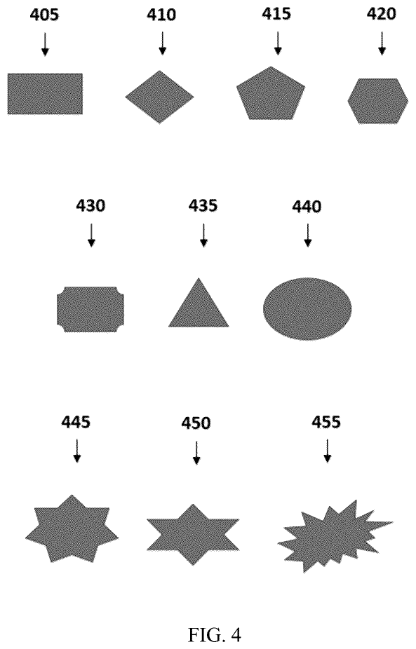

As shown in FIG. 1 and FIG. 2, for example, the first end segment ring (s) 115, the second end segment ring(s) 125, and the intermediate segment ring(s) 135 may be arranged in zig-zag or sinusoidal patterns. The frequencies of these patterns may be greater for the intermediate segment 130 than for the first end segment 110 and the second end segment 120. While zig-zag and sinusoidal strut patterns are shown in FIGS. 1 and 2, other strut patterns are also contemplated. As shown in FIG. 4, for example, the struts of the rings of the end and/or intermediate segments may have a rectangular pattern 405, a diamond pattern 410, a pentagon pattern 415, a hexagon pattern 420, an octagon pattern 430, a triangle pattern 435, an oval pattern 440, or a star-shaped pattern (such as a five pointed star, a six pointed star 450, a seven pointed star 445, or an more, such as a fourteen pointed star 455), or combinations thereof, to name a few.

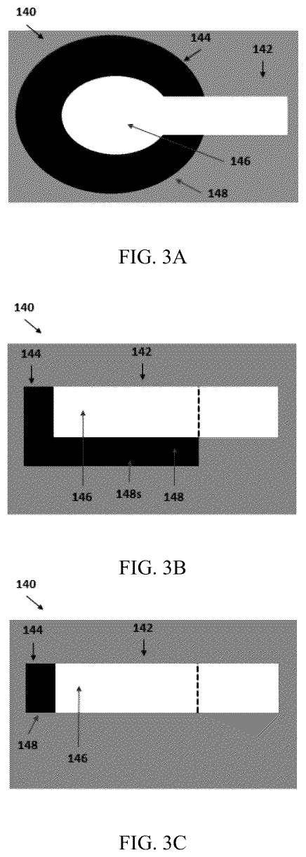

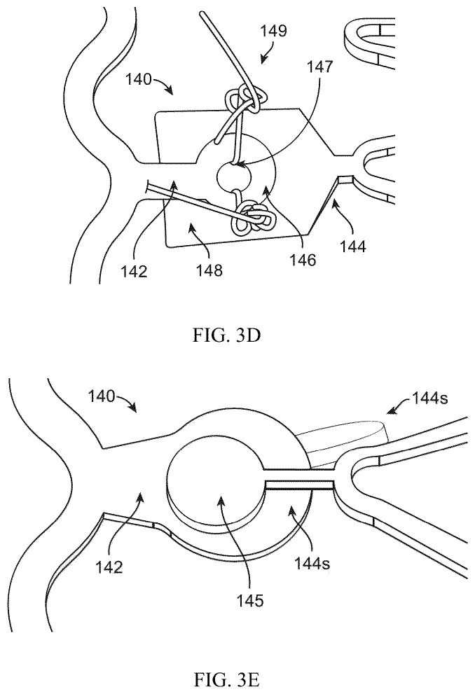

Referring back to FIGS. 1-2, as well as to FIGS. 3A-3E, the stent 100 may further comprise a plurality of connectors 140 connecting the first and second end segments 110, 120 to the intermediate segment 130. Each connector 140 may comprise a biodegradable connector arm 142 coupled to and extending away from the first or second end of the biodegradable intermediate segment 130, the biodegradable connector arm 142 having an end tab 146, and a non-biodegradable arm receiver 144 coupled to the end of the first or second end segment 110, 120 and configured to couple with the end tab 146 of the biodegradable connector arm 142. One or more of the connector arms 142 may have a length of 0.1 to 1 mm.

The plurality of connectors 140 may define a first connection segment between the end of the first end segment 110 and the first end of the intermediate segment 130 and a second connection segment between the end of the second end segment 120 and the second end of the intermediate segment 130. Such connection segments may be defined as the longitudinal area of the stent 100 between adjacent rings 115, 125 of the end segments 110, 120 and the rings 135 of the intermediate segment 130. One or both of the first and second connection segments may have a length of 0.5 to 1 mm.

In some embodiments, such as shown in FIG. 1 and magnified further in FIG. 3D, the non-biodegradable arm receiver 144 may comprise a socket 148 shaped to receive the end tab 146 of the biodegradable connector arm 142. In some embodiments, the end tab 146 may have an aperture 147 through which a suture or tether 149 or other structure may pass to further secure the end tab 146 to the non-biodegradable arm receiver 144, as shown in FIG. 1 and FIG. 3D.

In some embodiments, such as shown in FIG. 2 and magnified further in FIG. 3E, the non-biodegradable arm receiver 144 comprises a strut 144s configured to hook onto the end tab 146 of the biodegradable connector arm 142. In some embodiments, the end tab 146 may have an aperture 147 through which the strut 144s loops to secure the end tab 146, as shown in FIG. 1 and FIG. 3D. The strut 144s may be integral with a non-biodegradable tab 145 which may be shaped to fit at least partially within the aperture 147.

The connectors 140 may provide mortise and tenon joints between the non-biodegradable end segments 110, 120 and the biodegradable intermediate segment 150, which may prevent the lateral ends of the biodegradable end segment 150 from collapsing as its material degrades. FIGS. 3A-3C show schematic illustrations of an exemplary connector 140 in a side view (FIG. 3A) and section views orthogonal to the side view (FIGS. 3B, 3C). As shown in FIG. 3A, the end tab 146 of the connector arm 142 may have an ovoid shape that is sized to fit within the socket 148 of the receiver 144. The complementary shapes of the end tab 144 and the socket 148 may help prevent the end segments 110, 120 from de-coupling with the intermediate segment 130, particularly in the longitudinal direction of the stent 100 (i.e., parallel to the longitudinal axis of the stent 110), such as during deployment and expansion of the stent 100 as well as during the course of its implantation when the intermediate segment 130 may at least partially degrade or the stent 100 may be subject to movement of the vessel it is implanted in. While ovoid complementary shapes for the end tab 144 and the socket 148 are shown, other shapes, including ellipsoid, circular, polygonal, triangular, rectangular, pentagonal, hexagonal, or the like, may be used instead. In some embodiments, the thickness of the receiver 144 is greater than that of the connector arm 142 and the end tab 146, and the increased thickness can allow the socket 148 to have a support base 148s, as shown in FIG. 3B, which may help prevent the end-segments 110, 120 from de-coupling with the intermediate segment, particularly in the radial direction of the stent 100 (i.e., transverse to the longitudinal axis of the stent 110), such as during deployment and expansion of the stent 100 as well as during the course of its implantation when the intermediate segment 130 may at least partially degrade or the stent 100 may be subject to movement of the vessel it is implanted in. In some embodiments, the thickness of the receiver 144 is substantially the same as that of the connector arm 142 and the end tab 146, and a support base 148s is not provided, as shown in FIG. 3C.

While the connector arms 142 are described above as extending from and/or coupled to a longitudinal end of the intermediate segment 130 with the connector receivers 144 extending from and/or coupled to a longitudinal end of an end segment 110 or 120, the connector arm 142 and the connector receiver 144 may be reversed, alternatively or in combination. That is, connector arm(s) 142 may extend from and/or be coupled to a longitudinal end of an end segment 110 or 120, and the connector receivers 144s may extend from and/or be coupled to a longitudinal end of an intermediate segment 130.

Referring back to the stent 100, the first and second non-biodegradable end segments 110, 120 may be drug-eluting. For instance, the first and second non-biodegradable end segments 110, 120 may be at least partially coated with a therapeutic agent, such as an mTOR inhibitor, rapamycin, paclitaxel, sirolimus, or a combination thereof, to name a few. By providing non-biodegradable end segments 110, 120, a plurality of hybrid stents 100 may be overlapped or nested, making such stents 100 applicable for bifurcation lesions. The intermediate segment 130 may degrade over time, leaving the non-biodegradable end segments 110, 120 in place. Should restenosis occur, such as where the biodegraded intermediate segment 130 had been, a shorter stent may be implanted in its place.

The first and second non-biodegradable end segments 110, 120 may be made of a metal, such as stainless steel, cobalt chromium, or an alloy thereof. By being made of metal or other material highly visible to fluoroscopy or other medical imaging, the end segments 110, 120 can facilitate the accurate delivery and positioning of the stent 100.

The intermediate segment 130 may be made of a bioresorbable metal, metal alloy, or polymer, such as iron, magnesium, zinc, or an alloy thereof, in the case of a bioresorbable metal, or polylactic acid (PLA), poly-L-lactide (PLLA), or polylactic-co-glycolic acid (PLGA), in the case of a bioresorbable polymer.

The first end segment 110 may have a first length, the second end segment 120 may have a second length, and the intermediate segment 130 may have a third length greater than one or both of the first and second lengths. One or both of the lengths of the first end segment or the second end segment 110, 120 may be between 0.5 and 5 mm. The length of the intermediate segment 130 may be between 5 and 25 mm. One or both of the first and second end segments 110, 120 may have a thickness of 60 to 100 um. The intermediate segment may have a thickness of 50 to 120 um, for example 80 um.

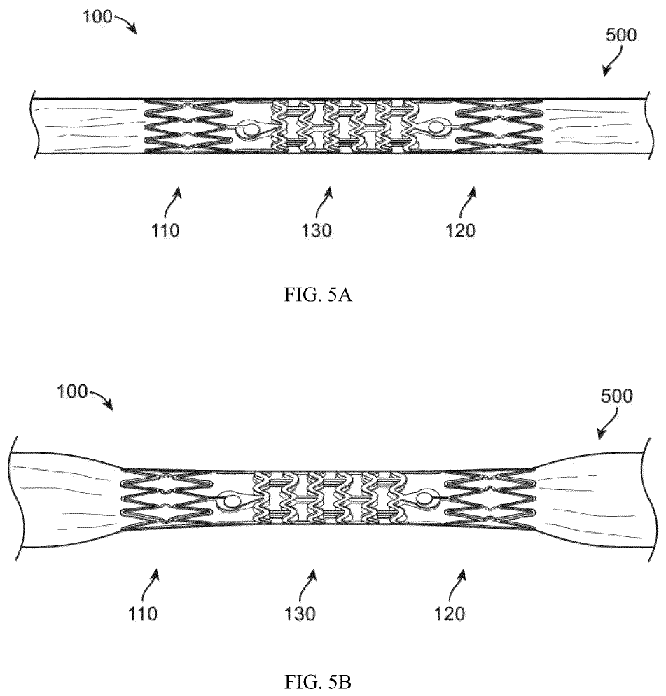

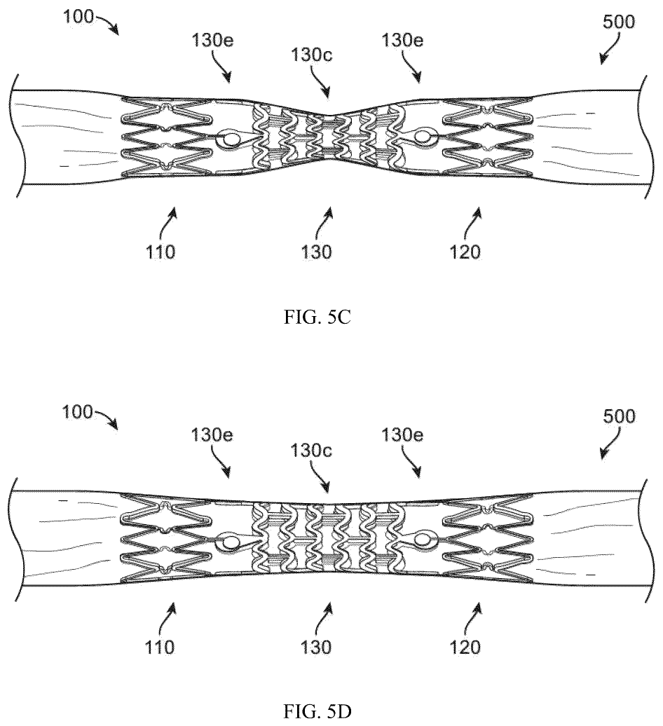

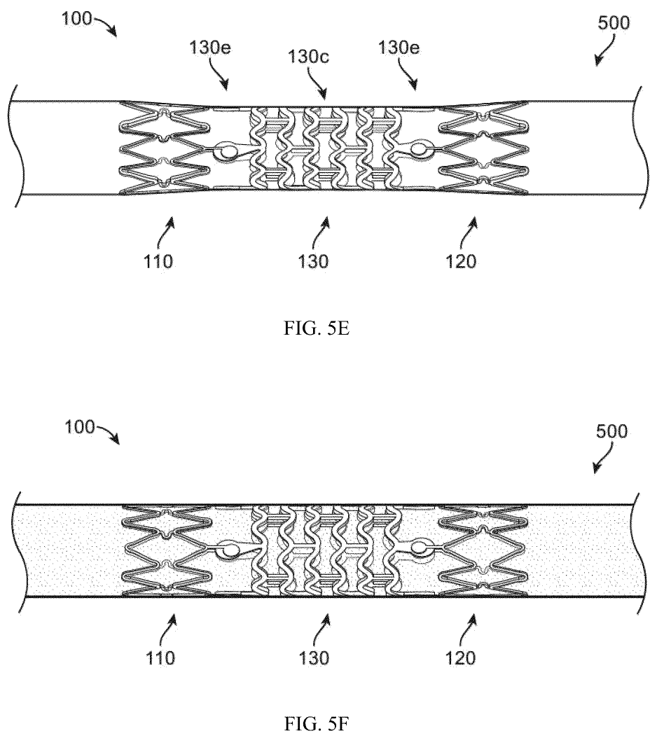

FIGS. 5A-5F show how the stent 100 may be deployed and expanded. As shown in FIG. 5A, the stent 100 may be crimped over an inflatable balloon 500 or other expandable member (such as a malecot) in a collapsed or undeployed configuration. As shown in FIG. 5B, as the balloon 500 begins to be inflated, the end segments 110, 120 may begin to expand while the intermediate segment 130 remains collapsed. As shown in FIG. 5C, as the balloon 500 is further inflated, the end segments 110, 120 may further expand, expanding along with them the end portions 130e of the intermediate segment 130, albeit not to the same extend as the end segments 110, 120, while the central portion 130c of the intermediate segment 130 remaining collapsed. As shown in FIG. 5D, as the balloon 500 is further inflated, the end segments 110, 120 may further expand, expanding along with it both end portions 130e and the central portion of the intermediate segment 130, albeit with the central portion 130c not expanded to the extent as the end portions 130e. As shown in FIG. 5E, as the balloon 500 is further inflated, the end segments 110, 120 and the intermediate segment 130 may now be expanded to substantially the same diameter. As shown in FIG. 5F, as the balloon 500 is further inflated to its maximum diameter, the stent 100 may now be expanded to its fullest extent, i.e., its the fully expanded or deployed configuration, including the send segments 110, 120 and the intermediate segment 130.

Although the above steps show a method of deploying a biodegradable drug-eluting stent in accordance with embodiments, a person of ordinary skill in the art will recognize many variations based on the teaching described herein. The steps may be completed in a different order.

Steps may be added or decided. Some of the steps may comprise sub-steps. Many of the steps may be repeated as often as beneficial or advantageous.

### Experimental Studies

A porcine-based animal study was conducted to compare bare metal stents (BMS), drug-eluting stents (DMSs), and hybrid stents according to embodiments herein (tested stent).

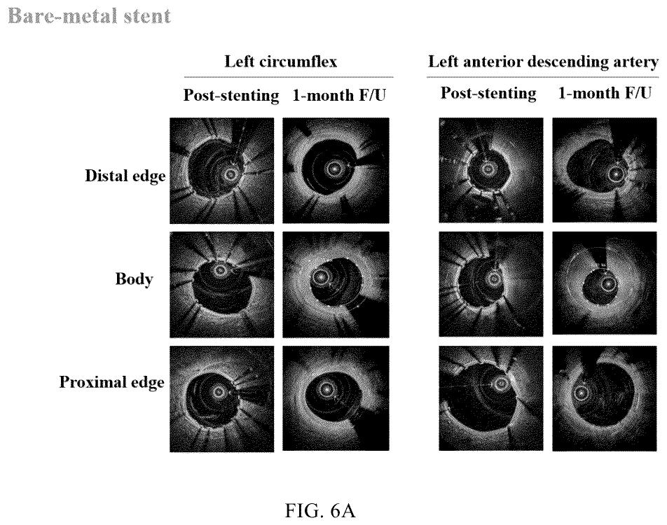

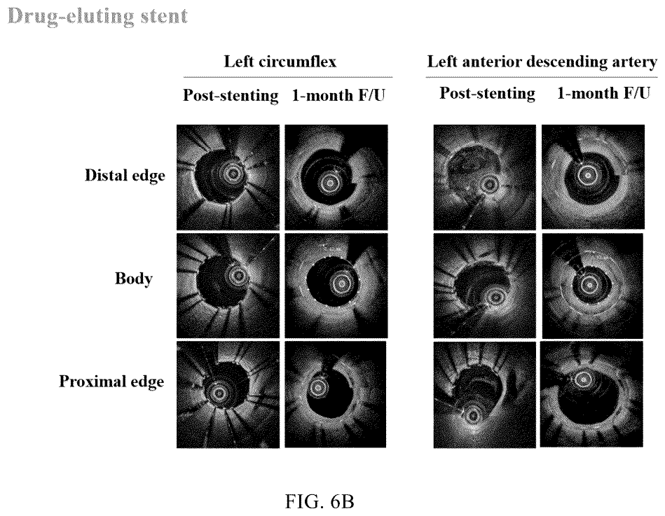

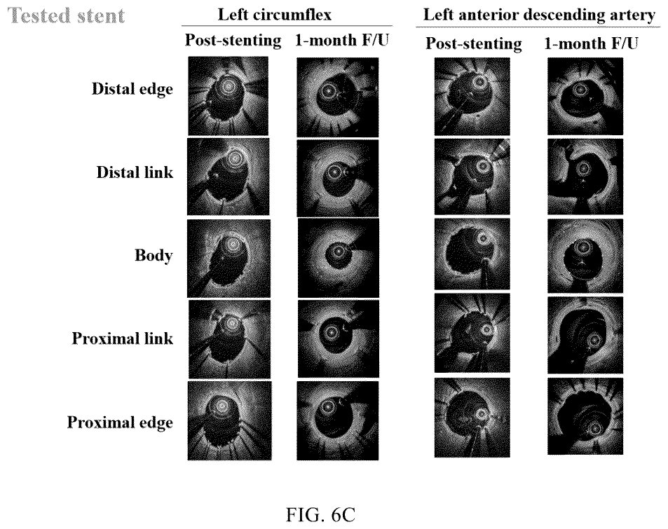

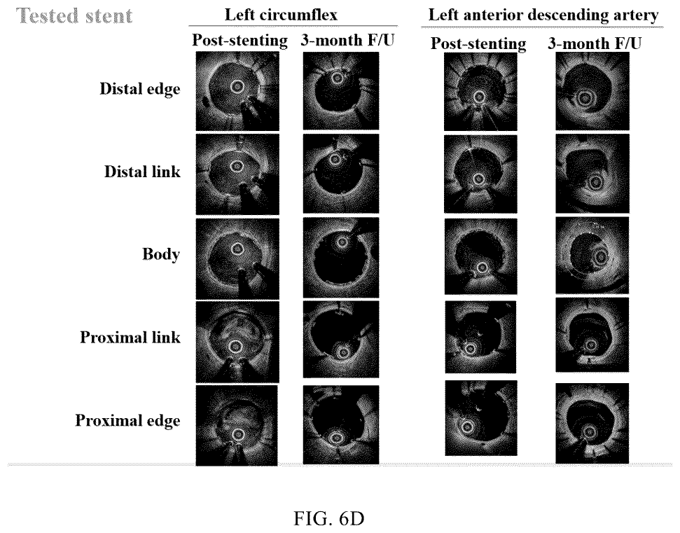

FIG. 6A shows optical coherence tomography (OCT) images of a left circumflex artery (LCX) and a left anterior descending artery (LAD) with a BMS deployed therein. The images were taken immediately after stent deployment and at a one month follow-up. The images were taken at the distal edge, body, and proximal edge of the stent. The stent struts can be observed as white arrows. For LCX, the BMS were well apposed immediately after stenting (post-stenting), without edge dissection or thrombus formation (similar to the findings from the QCA (quantitative comparative analysis) described below in reference to Table 1). At the one month follow-up, while both edges (proximal and distal) have mild proliferation, the stent body has severe intimal proliferation (as shown within the two circles marked on the respective image). For LAD, similarly to LCX, there were no post-procedure complications. At the one month follow-up, the proliferation at either proximal or distal edge was less severe than that in the LCX. However, the proliferation at the stent body (as shown within the two circles marked on the respective image) was significantly more severe than that in the LCX.

FIG. 6B shows optical coherence tomography (OCT) images of a left circumflex artery (LCX) and a left anterior descending artery (LAD) with DES deployed therein. The images were taken immediately after stent deployment and at a one month follow-up. The images were taken at the distal edge, body, and proximal edge of the stent. The stent struts can be observed as white arrows. For LCX, the DESs were well apposed immediately after stenting (post-stenting), without edge dissection, thrombus formation (similar to the findings from the QCA (quantitative comparative analysis) described below in reference to Table 1). At the one month follow-up, while both edges (proximal and distal) have mild proliferation, the stent body has severe intimal proliferation (as shown within the two circles marked on the respective image). For LAD, similarly to LCX, there were no post-procedure complications. At the one month follow-up, the proliferation at distal edge was less severe than that in the LCX. However, the proliferation at proximal edge and stent body (as shown within the two circles marked on the respective image) was significantly more severe than that in the LCX.

FIG. 6C shows optical coherence tomography (OCT) images of a left circumflex artery (LCX) and a left anterior descending artery (LAD) with a hybrid stent according to embodiments herein deployed therein (similar to the findings from the QCA described below in reference to Table 1). The images were taken immediately after stent deployment and at a one month follow-up. The images were taken at the distal edge, distal link (between the distal non-biodegradable portion and the biodegradable portion), body, proximal link (between the biodegradable portion and the proximal non-biodegradable portion), and proximal edge of the stent. The stent struts can be observed as white arrows. For LCX, the tested stent was well apposed immediately after stenting (post-stenting), without edge dissection, thrombus formation (as same as the finding from QCA). At the one month follow-up, proliferation at the edges, link segment, and stent body was similar to that found in the LCX and LAD. For LAD, similarly to LCX, there were no post-procedure complication. At the one month follow-up, the proliferation through the stent (from distal edge, distal link, body, proximal link to proximal edge) was less severe than that in the BMS and DES group.

Table 1 below shows a quantitative coronary analysis at the one month follow-up. The tested hybrid stent is associated with less in-stent restenosis as compared with BMS and DES. 13 pigs were studied in this experiment, with 3 in the bare-metal stent (BMS), 3 in the drug-eluting stent (DES) and 7 in the test stent groups. Both left circumflex (LCX) and left anterior descending artery (LAD) were stented in each pig using the same stent. There were no significant differences in stent length and stent diameter in either LCX or LAD between 2 groups. At one month follow-up, the tested hybrid stent is associated with less in-stent restenosis (mean value=9.8%) as compared with BMS (24.5%) and DES (31.0%, p=0.029) according to QCA analysis.

TABLE 1

| | BMS | DES | Tested stent | P |
|---|---|---|---|---|
| | (n = 3) | (n = 3) | (n = 7) | value |
| Left circumflex | | | | |
| Stent length, mm | 15.9 ± 1.5 | 13.5 ± 1.4 | 16.5 ± 2.5 | 0.188 |
| Stent diameter, mm | 2.55 ± 0.23 | 2.69 ± 0.24 | 2.32 ± 0.24 | 0.289 |
| Diameter stenosis, % | 15.5 ± 12.1 | 26.0 ± 9. 3 | 23.3 ± 6.9 | 0.185 |
| Left anterior descending | | | | |
| artery | | | | |
| Stent length, mm | 13.4 ± 0.8 | 17.4 ± 0.9 | 14.0 ± 2.6 | 0.346 |
| Stent diameter, mm | 2.56 ± 0.41 | 2.58 ± 0.12 | 2.55 ± 0.24 | 0.980 |
| Minimal lumen diameter, | 2.02 ± 0.35 | 1.18 ± 0.15 | 1.97 ± 0.57 | 0.069 |
| mm | 24.5 ± 12.0 | 31.0 ± 13.7 | 9.8 ± 6.9 | 0.029 |
| In-stent restenosis, % | | | | |

Table 2 below shows an analysis of OCT measurements at the one month follow-up. The tested hybrid stents tend to have a large lumen area when compared with BMS and DES, according to the OCT measurements. OCT measurements were made in 13 pigs (3 in BMS or DES group, 7 in tested stent group, respectively) one month after stenting procedure. The minimal stent area (MSA) measured in LCX by OCT was comparable between 3 groups. The MSA in the LAD in the tested hybrid stent group was significantly larger than 3.59±1.16 mm² in BMS and 3.39±0.27 mm² in DES groups (p=0.027).

TABLE 2

| | BMS | DES | Tested stent | P |
|---|---|---|---|---|
| | (n = 3) | (n = 3) | (n = 7) | value |
| Left circumflex | | | | |
| Minimal lumen area, mm² | 3.09 ± 1.13 | 2.81 ± 1.0 | 3.02 ± 1.15 | 0.088 |
| Thrombus, n (%) | 0 | 0 | 0 | |
| Left anterior descending | | | | |
| artery | | | | |
| Minimal lumen area, mm² | 3.59 ± 1.16 | 3.39 ± 0.27 | 4.19 ± 0.53 | 0.027 |
| Thrombus, n (%) | 0 | 0 | 0 | |

Table 3 below shows an analysis of OCT measurements at a three month follow-up. FIG. 6D shows the OCT images themselves, i.e., images of a left circumflex artery (LCX) and a left anterior descending artery (LAD) with a hybrid stent according to embodiments herein deployed therein, immediately post-tenting and at the three-month follow-up. The images were taken at the distal edge, distal link (between the distal non-biodegradable portion and the biodegradable portion), body, proximal link (between the biodegradable portion and the proximal non-biodegradable portion), and proximal edge of the stent. As shown by the images of FIG. 6D and Table 6D, a large minimal lumen area was maintained through the three month follow-up period, with a lower late lumen loss from OCT measurements in both LCX and LAD.

TABLE 3

| | Tested stent |
|---|---|
| | (n = 3) |
| Left circumflex | |
| Minimal lumen area, mm² | 2.52 ± 0.55 |
| late lumen loss, mm² | 0.23 ± 0.32 |
| Thrombus, n (%) | 0 |
| Left anterior descending | |
| artery | |
| Minimal lumen area, mm² | 3.66 ± 0.63 |
| late lumen loss, mm² | 0.18 ± 0.24 |
| Thrombus, n (%) | 0 |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

1.-22. (canceled)

23. A stent comprising:
a first non-biodegradable end segment;
a biodegradable intermediate segment having a first end coupled to an end of the first non-biodegradable end segment and a second end;
a second non-biodegradable end segment having an end coupled to the second end of the biodegradable intermediate segment; and
a plurality of connectors connecting the first and second end segments to the intermediate segment.

24. The stent of claim 23, wherein each connector comprises:
a biodegradable connector arm coupled to and extending away from the first or second end of the biodegradable intermediate segment, the biodegradable connector arm having an end tab, and
a non-biodegradable arm receiver coupled to the end of the first or second end segment and configured to couple with the end tab of the biodegradable connector arm.

25. The stent of claim 24, wherein the plurality of connectors define a first connection segment between the end of the first end segment and the first end of the intermediate segment and a second connection segment between the end of the second end segment and the second end of the intermediate segment.

26. The stent of claim 25, wherein one or both of the first and second connection segments have a length of 0.5 to 1 mm.

27. The stent of claim 24, wherein one or more of the connector arms has a length of 0.1 to 1 mm.

28. The stent of claim 24, wherein the non-biodegradable arm receiver comprises a socket shaped to receive the end tab of the biodegradable connector arm.

29. The stent of claim 24, wherein the non-biodegradable arm receiver comprises a strut configured to hook onto the end tab of the biodegradable connector arm.

30. The stent of claim 23, wherein the first and second end segments are configured to expand at a first rate in response to an expansion force, wherein the intermediate segment is configured to expand at a second rate in response to the expansion force, and wherein the first rate is faster than the second rate.

31. The stent of claim 23, wherein the first end segment comprises at least one ring of struts arranged in a first non-biodegradable strut pattern, wherein the second end segment comprises at least one ring of struts arranged in a second non-biodegradable strut pattern, wherein the intermediate segment comprises at least one ring of struts arranged in a biodegradable strut pattern, and wherein the biodegradable strut pattern of the intermediate segment is denser than the first and second non-biodegradable strut patterns of the first and second non-biodegradable end segments.

32. The stent of claim 31, wherein the at least one ring of struts of the first or second end segments comprises a plurality of non-biodegradable struts arranged in a first zig-zag or sinusoidal pattern with a first frequency, and wherein the at least one ring of struts of the intermediate segment comprises a plurality of biodegradable struts arranged in a second zig-zag or sinusoidal pattern with a second frequency greater than the first frequency.

33. The stent of claim 23, wherein the first and second non-biodegradable end segments are drug-eluting.

34. The stent of claim 33, wherein the first and second non-biodegradable end segments are at least partially coated with a therapeutic agent.

35. (canceled)

36. The stent of claim 23, wherein the first and second non-biodegradable end segments are made of a metal.

37. (canceled)

38. The stent of claim 23, wherein the intermediate segment is made of a bioresorbable metal, metal alloy, or polymer.

39. (canceled)

40. (canceled)

41. The stent of claim 23, wherein the stent is balloon-expandable.

42. The stent of claim 23, wherein the first end segment has a first length, the second end segment has a second length, and the intermediate segment has a third length greater than one or both of the first and second lengths.

43. The stent of claim 23, wherein one or both of the lengths of the first end segment or the second end segment is between 0.5 and 5 mm.

44. The stent of claim 23, wherein the length of the intermediate segment is between 5 and 25 mm.

45. The stent of claim 23, wherein one or both of the first and second end segments has a thickness of 60 to 100 um.

46. The stent of claim 23, wherein the intermediate segment has a thickness of 50 to 120 um.
